(19) United States
(12) Patent Application Publication
(10) Pub. No.: US 2025/0197894 A1
(43) Pub. Date: Jun. 19, 2025

(54) PROCESS AND/OR FACILITY INTEGRATION OF ANAEROBIC DIGESTION OF ONE OR MORE STILLAGE COMPOSITIONS

(71) Applicant: POET Research, Inc., Sioux Falls, SD (US)

(72) Inventors: David Charles Carlson, Brandon, SD (US); Ryan Wood, Sioux Falls, SD (US); Brady Christopher Clavel, Yankton, SD (US)

(21) Appl. No.: 18/978,338

(22) Filed: Dec. 12, 2024

Related U.S. Application Data
(60) Provisional application No. 63/610,702, filed on Dec. 15, 2023.

(51) Int. Cl. C12P 5/02; C01C 1/02; C12F 3/10

(52) U.S. Cl. CPC C12P 5/023; C01C 1/02; C12F 3/10

(57) ABSTRACT

A bioprocessing facility that includes: a source of one or more stillage compositions; a multi-effect evaporator system including at least a final-effect evaporator; and an anaerobic digestion system in fluid communication with the final-effect evaporator. The final-effect evaporator is configured to receive at least a portion of at least one stillage composition as feed. The final-effect evaporator is also configured to produce a vapor composition and a concentrated stillage composition. The anaerobic digestion system is configured to receive and digest at least a portion of the concentrated stillage composition to produce a biogas and an anaerobic digestion digestate composition. The multi-effect evaporator system includes at least one effect evaporator prior to the final-effect evaporator that is configured to receive at least a portion of the anaerobic digestion digestate composition as feed. Related methods of producing biogas from one or more stillage compositions.

### CROSS-REFERENCE TO RELATED APPLICATION

This nonprovisional patent application claims the benefit of U.S. Provisional Patent Application Ser. No. 63/610,702, filed on Dec. 15, 2023, wherein said provisional patent application is incorporated herein by reference in its entirety.

### BACKGROUND

The present disclosure relates to methods and systems that produce stillage compositions and using one or more stillage compositions as feed for anaerobic digestion.

There is a continuing need for new and improved methods and/or systems for incorporating anaerobic digestion into bioprocessing facilities that produce stillage compositions.

### SUMMARY

Embodiments of the present disclosure include a bioprocessing facility that includes: a source of one or more stillage compositions; a multi-effect evaporator system including at least a final-effect evaporator; and an anaerobic digestion system in fluid communication with the final-effect evaporator. The final-effect evaporator is configured to receive at least a portion of at least one stillage composition as feed, The final-effect evaporator is configured to produce a vapor composition and a concentrated stillage composition. The anaerobic digestion system is configured to receive and digest at least a portion of the concentrated stillage composition to produce a biogas and an anaerobic digestion digestate composition. The multi-effect evaporator system includes at least one effect evaporator prior to the final-effect evaporator that is configured to receive at least a portion of the anaerobic digestion digestate composition as feed.

Embodiments of the present disclosure also include a method of producing biogas from one or more stillage compositions. The method includes introducing at least a portion of at least one stillage composition as feed into a final-effect evaporator to form a vapor composition and a concentrated stillage composition; exposing at least a portion of the concentrated stillage composition to anaerobic digestion conditions to produce a biogas and an anaerobic digestion digestate composition; and introducing at least a portion of the anaerobic digestion digestate composition as feed into at least one effect evaporator prior to the final-effect evaporator to form a vapor composition and a concentrated anaerobic digestion digestate composition.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1 is a schematic showing an illustrative embodiment of a bioprocessing facility that generates stillage compositions from beer;

FIG. 2 illustrates non-limiting embodiments according to the present disclosure of incorporating anaerobic digestion into the bioprocessing facility 100 shown in FIG. 1.

FIG. 3 illustrates another non-limiting embodiment according to the present disclosure of incorporating anaerobic digestion into the bioprocessing facility 100 shown in FIG. 1;

FIG. 4 illustrates another non-limiting embodiment according to the present disclosure of incorporating anaerobic digestion into the bioprocessing facility 100 shown in FIG. 1; and

FIG. 5 illustrates another non-limiting embodiment according to the present disclosure of incorporating anaerobic digestion into the bioprocessing facility 100 shown in FIG. 1.

### DETAILED DESCRIPTION

The present disclosure relates to integrating one or more anaerobic digestion systems into a bioprocessing facility that produces one or more stillage compositions as a byproduct/co-product. As used herein, a “bioprocessing facility” refers to a facility that can produce one or more bioproducts by converting biomass feedstock via one or more physical processes, one or more chemical processes, one or more bioprocesses, and combinations thereof. Non-limiting examples of bioprocessing facilities include dry mills, wet mills, biofuel production facilities, pharmaceutical production facilities, soy processing facilities, breweries, bakeries, and the like.

A bioproduct refers to a product derived from a biological, renewable resource. For example, a bioproduct can be a component of biomass feedstock (e.g., grain feedstock) that is liberated from the biomass feedstock (e.g., grain oil such as corn oil from corn grain) and/or can include a chemical (“biochemical” or “target biochemical”) that is produced by a biocatalyst (e.g., microorganism and/or enzyme) such as, for example, alcohol produced by yeast fermenting sugar. Non-limiting examples of bioproducts produced in a bioprocessing facility include one or more of fuel, feed, food, pharmaceuticals, beverages and precursor chemicals. In some embodiments, a bioproduct includes, among others, one or more monomeric sugars, one or more enzymes, one or more oils, one or more alcohols (e.g., ethanol, butanol, and the like), fungal biomass, amino acids, and one or more organic acids (e.g., lactic acid), and combinations thereof.

In some embodiments, one or more bioprocesses are carried out in a bioprocessing facility utilizing living cells (one or more microorganisms) and/or their components (e.g., enzymes produced by a microorganism) to obtain a desired bioproduct. Non-limiting examples of bioprocesses include one or more of hydrolysis (e.g., enzymatic hydrolysis), aerobic fermentation, or anaerobic fermentation. In some embodiments, a bioprocess includes saccharification and fermentation of a plant-based feedstock into a biofuel via enzymatic hydrolysis and yeast-based fermentation of the hydrolysate (e.g., yeast-based fermentation in a grain-to-ethanol biofuel facility).

One or more bioproducts can be separated from beer to form at least one target bioproduct stream (e.g., ethanol) and one or more co-product streams (e.g., whole stillage). A co-product stream can encompass any stillage composition downstream from fermentation after separating one or more bioproducts from beer using one or more thermal-based separation technologies such as distillation, evaporation, and the like. As used herein, a “stillage composition” can include whole stillage, at least one stillage composition derived from whole stillage, and combinations thereof. Non-limiting examples of a stillage composition derived from whole stillage include wet cake, thin stillage, concentrated thin stillage (syrup), defatted syrup, defatted emulsion, clarified thin stillage, distiller's oil, distiller's grain, distiller's yeast, and the like. Non-limiting examples of methods and systems for processing stillage streams are also described in U.S. Pat. No. 8,702,819 (Bootsma); U.S. Pat. No. 9,061,987 (Bootsma); U.S. Pat. No. 9,290,728 (Bootsma); U.S. Pat. No. 10,059,966 (Bootsma); U.S. Pat. No. 11,248,197 (Bootsma); and U.S. Pub. No. 2020/0140899 (Bootsma); wherein the entirety of each of said patent documents is incorporated herein by reference.

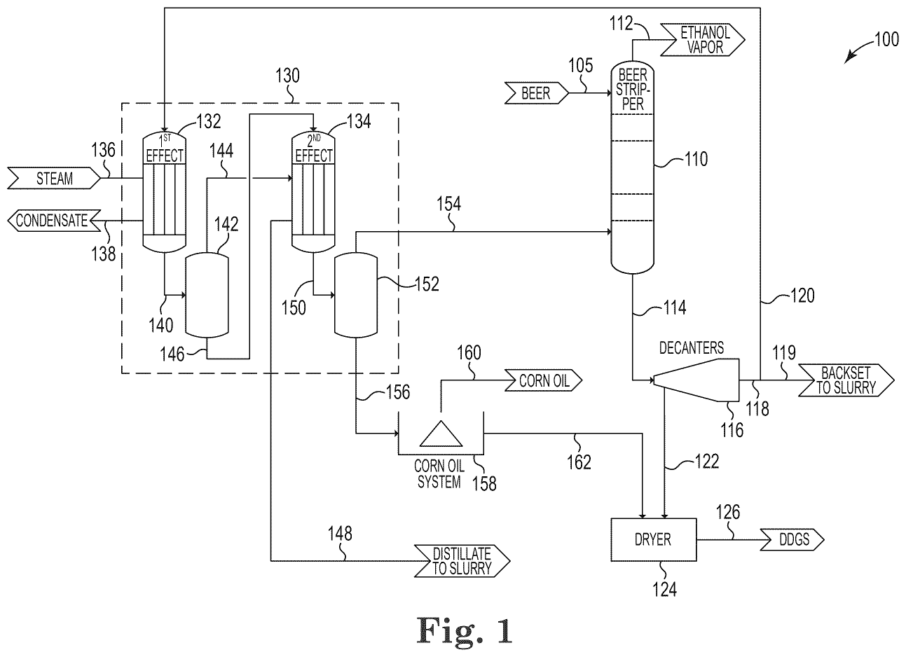

FIG. 1 shows a non-limiting example of a bioprocessing facility 100 that forms a beer 105, e.g., in a dry-grind corn ethanol bioprocessing facility. As shown in FIG. 1, bioprocessing facility 100 produces ethanol as a biochemical 112 that is separated from beer 105 via a distillation system 110 to produce whole stillage 114. FIG. 1 shows the distillation column of distillation system 110, which can also include a reflux section (not shown) and a reboiler portion (not shown). Whole stillage is separated in separation system 116 into thin stillage 118 and wet cake 122. Separation system can include a wide variety of one or more solid-liquid separators to separate whole stillage into thin stillage 118 and wet cake 122. Non-limiting examples of solid-liquid separators include one or more centrifuges (e.g., two-phase vertical disk-stack centrifuge, three-phase vertical disk-stack centrifuge, filtration centrifuge), one or more decanters (e.g., filtration decanters), one or more filters (e.g., fiber filter, rotary vacuum drum filter, filter device having one or more membrane filters), one or more screen devices (e.g., a “DSM” screen; one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, and combinations thereof. As shown in FIG. 1, separation system 116 includes one or more decanters.

As shown, a portion 120 of thin stillage 118 is sent to be concentrated in a multi-effect evaporator system 130. Optionally, a portion 119 portion of thin stillage 118 can be recycled upstream as backset to form a slurry of ground corn for fermentation (not shown).

A multi-effect evaporator system includes two or more evaporators in series operating at different pressures such that the vapor generated in one of the effects can be used as a heat source and condense in the subsequent effect. In this way a multi-effect evaporator system can perform a continuous evaporation on an initial “feed” stream to concentrate the feed stream. It is noted that one or more evaporators could be included in a multi-effect evaporator system to operate as multiple stages of evaporators in parallel with a given “effect” evaporator. One non-limiting example of an evaporator is a falling film evaporator, which includes a vertical shell and tube heat exchanger. A liquid feed stream can be continuously recirculated to the top of the “tube side” and form a film on the inner surface of the tube as it falls from the top to the bottom under the force of gravity. Each evaporator has a vapor stream as a heat source that enters on the “shell side” of the evaporator to transfers heat to a feed stream that is fed on a “tube side” of the evaporator. The vapor stream indirectly heats the feed stream such that the vapor stream at least partially condenses to form a condensate. An “effect” is defined as operating at a given boiling temperature of the feed stream to concentrate the feed stream. For example, a stillage composition can be fed to an evaporator of a given “effect” to form a concentrated stillage composition. A downstream “effect” evaporator, or subsequent “effect” evaporator (e.g., “final-effect evaporator) operates a lower boing temperature (e.g., by operating at a lower pressure). Likewise a subsequent “effect” evaporator can concentrate the stream that is fed to it. The heating section of an evaporator may be the same or separate from the vapor/liquid separation section. As shown in FIGS. 1-5, the heating section is separate from a flash tank where vapor/liquid separation occurs.

In more detail, portion 120 is fed as feed to the tube side of a first-effect evaporator 132, is illustrated as a falling film evaporator. The portion 120 is heated via a stream of steam 136 on the shell side of first-effect evaporator 132. Portion 120 is heated by steam 136, which condenses into condensate 138. Heated thin stillage 140 is sent to flash tank 142 so that it can separate into vapor composition 144 and partially, concentrated thin stillage 146. In some embodiments, a portion of the concentrated thin stillage 146 may optionally be recirculated to the inlet of the first effect evaporator 132 to maintain sufficient liquid flow through the evaporator (not shown). The concentrated thin stillage 146 is sent to the tube side of final-effect evaporator 134 (which is a second-effect evaporator in this embodiment) and the vapor composition 144 is fed as a heat source to the shell side of final-effect evaporator 134, which is illustrated as a falling film evaporator. The partially, concentrated thin stillage 146 is heated by vapor composition 144, which condenses into condensate 148. As shown, condensate 148 (also referred to as distillate) can be recycled upstream to form a slurry with ground corn for fermentation. The heated, partially, concentrated thin stillage 150 is sent to flash tank 152 so that it can separate into vapor composition 154 and concentrated thin stillage 156. As shown in FIG. 1, the vapor composition 154 is introduced into the bottom “stripping” section of distillation column of distillation system 110 to provide heat to distillation system 110.

The concentrated thin stillage 156 can be sent to a separator system 158 to separate corn oil 160 and defatted thin stillage 162 from concentrated thin stillage 156. In some embodiments, a portion of the concentrated thin stillage 156 may optionally be recirculated to the inlet of the second effect evaporator 134 to maintain sufficient liquid flow through the evaporator (not shown).

As shown in FIG. 1, wet cake 122 and defatted thin stillage are combined and dried in dryer system 124 to form dried distillers' grain with solubles (DDGS) 126.

According to the present disclosure an anaerobic digestion system is incorporated into a bioprocessing facility, such as a dry-grind corn facility, to use one or more stillage compositions as feed to form biogas. Advantageously, the anaerobic digestion system can be incorporated in a manner that manages one or more of energy balance and/or water balance of the bioprocessing facility while at the same time managing ammonia that is produced during anaerobic digestion so that it does not impact one or more processes or bioproducts of the bioprocessing facility.

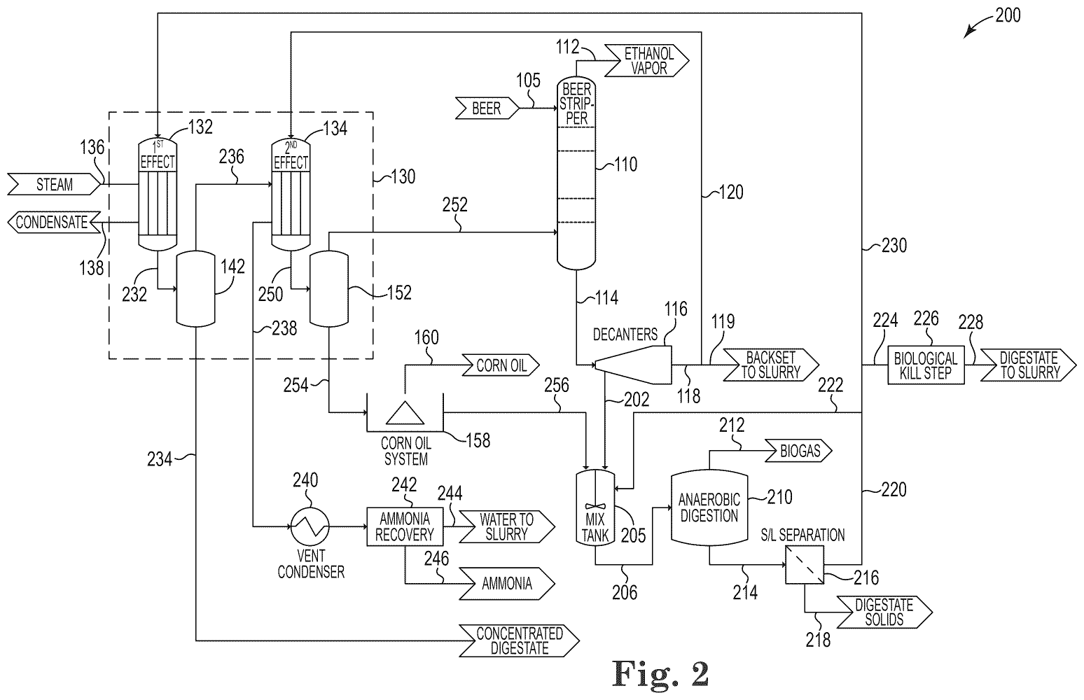

FIGS. 2-5 each illustrate non-limiting embodiments according to the present disclosure of incorporating anaerobic digestion into the bioprocessing facility 100 shown in FIG. 1. Reference characters discussed above with respect to FIG. 1 may be repeated in one or more of FIGS. 2-5 to illustrate the same or similar system or process stream as shown in FIG. 1. Reference characters repeated in any of FIGS. 2-5 may or may not be discussed again.

As described below, the bioprocessing facility incorporates anaerobic digestion of whole stillage in a manner that recycles energy and water in bioprocessing facility 200 while at the same time managing ammonia produced in anaerobic digestion relative to recycling process streams in the bioprocessing facility for water and/or energy balance purposes.

As shown in FIG. 2, portion 120 of thin stillage 118 is introduced as feed into the final-effect evaporator 134, which is a second-effect evaporator in this embodiment. The heated thin stillage 250 is sent to flash tank 152 so that it can separate into vapor composition 252 and concentrated thin stillage 254. Alternatively, final-effect evaporator 134 could be a third-effect, fourth-effect, etc. evaporator depending on the desired number of “effects” for the multi-effect evaporator system. For example, managing the water balance of a bioprocessing facility such as bioprocessing facility 200 can influence the number of “effects” selected. By introducing portion 120 of thin stillage 118 as feed into the final-effect evaporator 134 the vapor composition 252 that is generated in the final-effect evaporator 134 and injected into the bottom of the beer stripper of the distillation system 110 for thermal energy is free of ammonia and is similar to the vapor composition 154 coming from the final-effect evaporator 134 in FIG. 1.

Corn oil is removed from the concentrated thin stillage 254 coming from the final-effect evaporator 134. Removing corn oil from concentrated thin stillage 254 before anaerobic digestion can reduce or mitigate inhibition caused by corn oil during anaerobic digestion. Also, in some embodiments, separating corn oil permits it to be sold as a higher-value co-product instead of being converted to biogas. Finally, by concentrating the portion 120 of thin stillage 118 in at least the final-effect evaporator 134, water can be removed to form concentrated thin stillage 254 having a relatively lower flow rate so that the separator system 158 can operate more efficiently.

The defatted, concentrated thin stillage 256 is mixed with the wet cake 202 from the decanters of the separation system 116 in mix tank 205, and the mixture (aka “defatted whole stillage”) 206 is fed to the anaerobic digestion system 210, where it is exposed to anaerobic digestion conditions. The anaerobic digestion system 210 is configured to digest at least a portion of the mixture 206 to produce a biogas 212 and an anaerobic digestion digestate composition 214.

A separation system 216 is in fluid communication with the anaerobic digestion digestate composition 214 and is configured to separate the anaerobic digestion digestate composition 214 into at least an anaerobic digestion liquid effluent 220 and an anaerobic digestion solid effluent 218. The anaerobic digestion solid effluent 218, which includes most of the suspended solids in the anaerobic digestion digestate composition 214, can be applied to land in some embodiments if desired.

A multi-effect evaporator system according to the present disclosure includes at least one effect evaporator prior to the final-effect evaporator 134 that is configured to receive a portion 230 of the anaerobic digestion liquid effluent 220 as feed. As illustrated in FIG. 2, portion 230 of the anaerobic digestion liquid effluent 220 is fed to first-effect evaporator 132 where it is concentrated. The heated, portion 232 of the anaerobic digestion liquid effluent 220 is sent to flash tank 142 so that it can separate into vapor composition 236 and a concentrated anaerobic digestion digestate composition such as concentrated anaerobic digestion liquid effluent 234. In some embodiments, the concentrated anaerobic digestion liquid effluent 234 is stored and land applied as a fertilizer. The vapor composition 236, which includes ammonia rich vapors, are condensed on the shell of the final-effect evaporator 134 as condensate 238 and do not enter the distillation system 110, where the ammonia could cause potential contamination issues of biochemical 112 (e.g., ethanol). As can be seen, the energy in vapor composition 236 can be captured in a manner using existing equipment/systems in a bioprocessing facility such as multi-effect evaporator system 130 that does not risk ammonia contamination of the biochemical 112 product. Optionally, a separate condenser 240 may be used to cool condensate 238 and ensure that all of the ammonia has completely condensed.

The condensate 238 (ammonia rich distillate) can be processed by one of several potential unit operations in ammonia separation system 242 to separate an ammonia portion 246 from the water portion 244. The relative purity of the ammonia portion 246 and water portion 244 depends of the ammonia separation system 242. In some embodiments, the water can be recycled upstream to form a slurry with feedstock (e.g., ground corn) for fermentation and the ammonia can be sold as a co-product (for example, to be used as a fertilizer). As can be seen, the water balance of bioprocessing facility 200 can be managed even though ammonia is generated in anaerobic digestion system 210 and existing equipment/systems in a bioprocessing facility such as multi-effect evaporator system 130 is used to separate ammonia from anaerobic digestion liquid effluent 220.

Optionally, a portion 222 of the anaerobic digestion liquid effluent 220 is recycled to the anaerobic digestion system 210 to mix tank 205 to dilute the solids concentration and reduce the viscosity of the mixture 206 if desired. As another option, a portion (not shown) of the anaerobic digestion digestate composition 214 may be used as dilution liquid instead of, or in addition to, the portion 222 of the anaerobic digestion liquid effluent 220 as just discussed. As yet another option, a portion (not shown) of the water portion 244 may be used as dilution liquid instead of, or in addition to, the portion 222 and/or the portion (not shown) of the anaerobic digestion digestate composition 214 as just discussed.

Optionally, a portion 224 of the remaining liquid digestate is optionally recycled to slurry as makeup water 228. This may be done in place of or in addition to thin stillage being sent to slurry as backset. The digestate recycled to slurry may go through an optional biological kill step 226 prior to being added to the slurry step in order to reduce the risk of infection and contamination of the starch to ethanol fermentation. The biological kill step may consist of a thermal shock system (high temperature and short time sterilization or pasteurization step), an evaporation step, a membrane filtration step, or any other method to substantially reduce the concentration of live organisms in the recycled digestate.

In some embodiments, where the anaerobic digestion solid effluent 218 and/or the concentrated anaerobic digestion liquid effluent 234 have a relatively high solids content, there may be a relatively higher volumetric flowrate of water portion 244 as compared to condensate 148. In some embodiments, the volumetric flowrate of water portion 244 may by more than the slurry system in front end of bioprocessing facility 200 is capable of using. In these cases, a portion of water portion 244 may be processed through additional unit operations (such as RO membrane filtration, aerobic digestion treatment, or potentially no additional unit operations) and/or used as makeup water in the cooling tower.

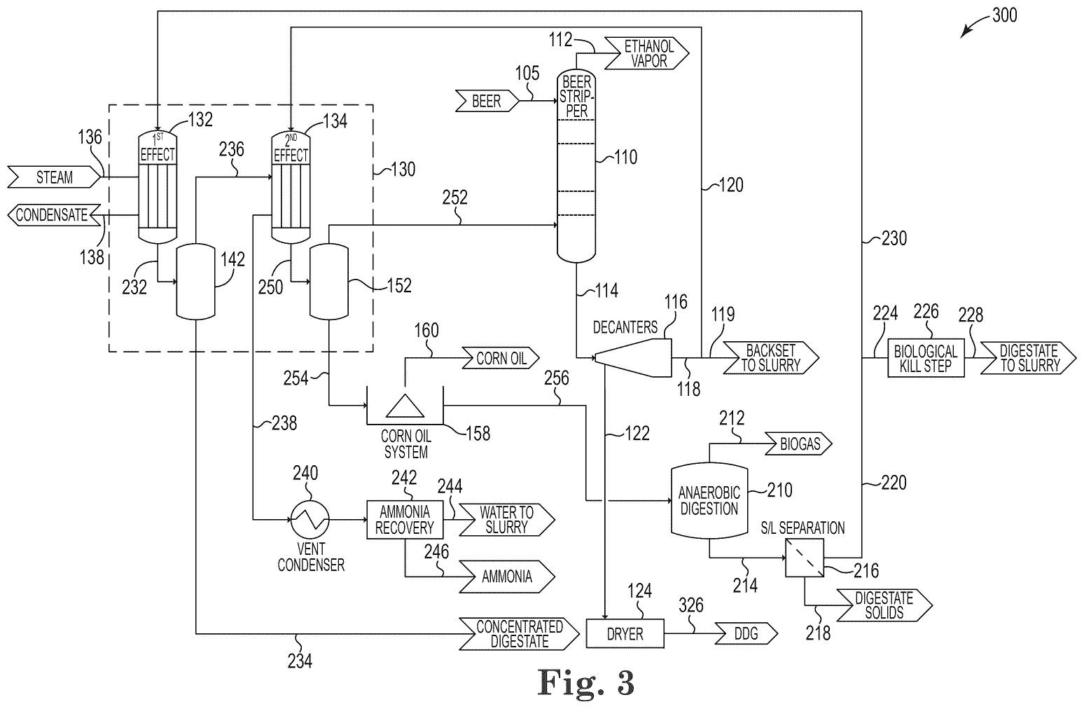

FIG. 3 shows an alternative bioprocessing facility 300 that incorporates anaerobic digestion. Bioprocessing facility 300 is similar to bioprocessing facility 200 in FIG. 2, described above, except that wet cake 122 is dried in dryer system 124 to produce dried distillers' grain (DDG) 326 instead of being sent to mix tank 205 and anaerobic digestion system 210. It is noted that because wet cake is not sent to anaerobic digestion system 210, the feed to anaerobic digestion system 210 will have lower total solids, especially lower suspended solids. In some embodiments, because of the lower solids being fed to anaerobic digestion system 210, a separation system 216 may not be needed to separate anaerobic digestion digestate composition 214 and the anaerobic digestion digestate composition 214, or fraction thereof, may be sent to first-effect evaporator 132.

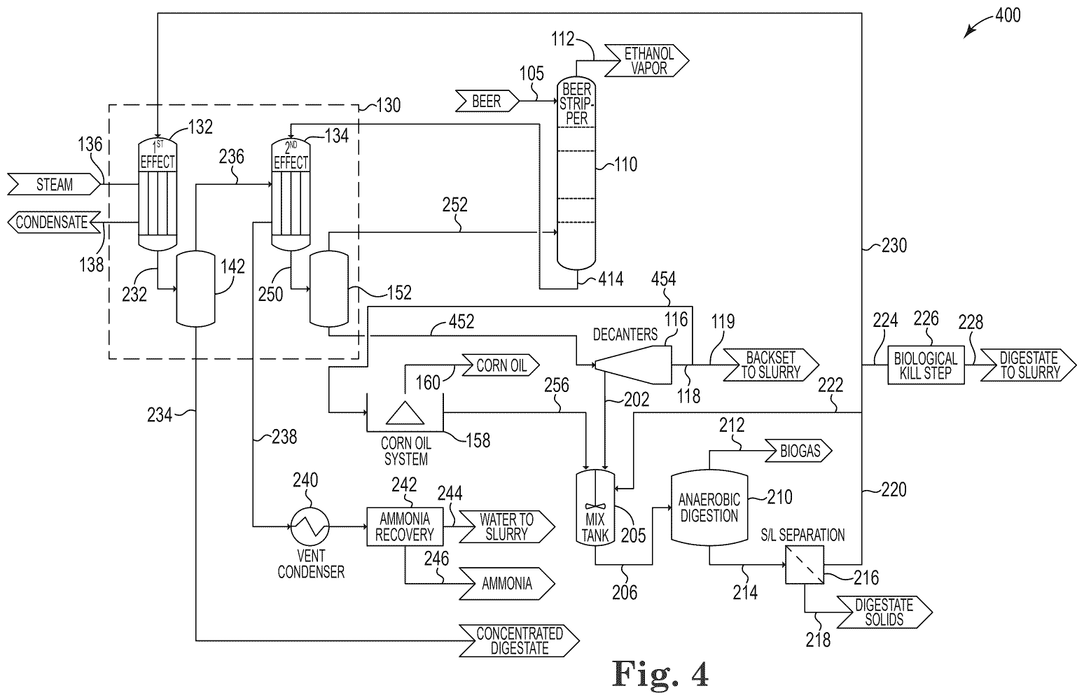

FIG. 4 shows another alternative bioprocessing facility 400 that incorporates anaerobic digestion. Bioprocessing facility 400 is similar to bioprocessing facility 200 in FIG. 2, described above, except that whole stillage 414 is sent to final-effect evaporator 134 instead of first being separated into thin stillage 118 and wet cake 122. This tends to reduce the hydraulic loading rate of the separation system 116 (e.g., decanters), but may increase the fouling rate in the final-effect evaporator 134 due to the higher solids content of whole stillage 414.

Referring to FIG. 4, the concentrated whole stillage 452 from flash tank 152 is then separated in separation system 116 into thin stillage 118 and wet cake 202. A portion 454 of thin stillage 118 is sent directly to separator system 158 instead of final-effect evaporator 134 first like in FIGS. 2 and 3. At least corn oil 160 and defatted thin stillage 256 are separated from portion 454 in separator system 158.

In some embodiments, the concentration of the feed to the anaerobic digestion system 210 can be adjusted as desired. For example, as the feed or stillage compositions used to make the feed are more concentrated, the concentration of salts, produced ammonia, and all other compounds in the anaerobic digestion system, can be relatively more concentrated, some of which may be inhibitory to the anaerobic digestion biology.

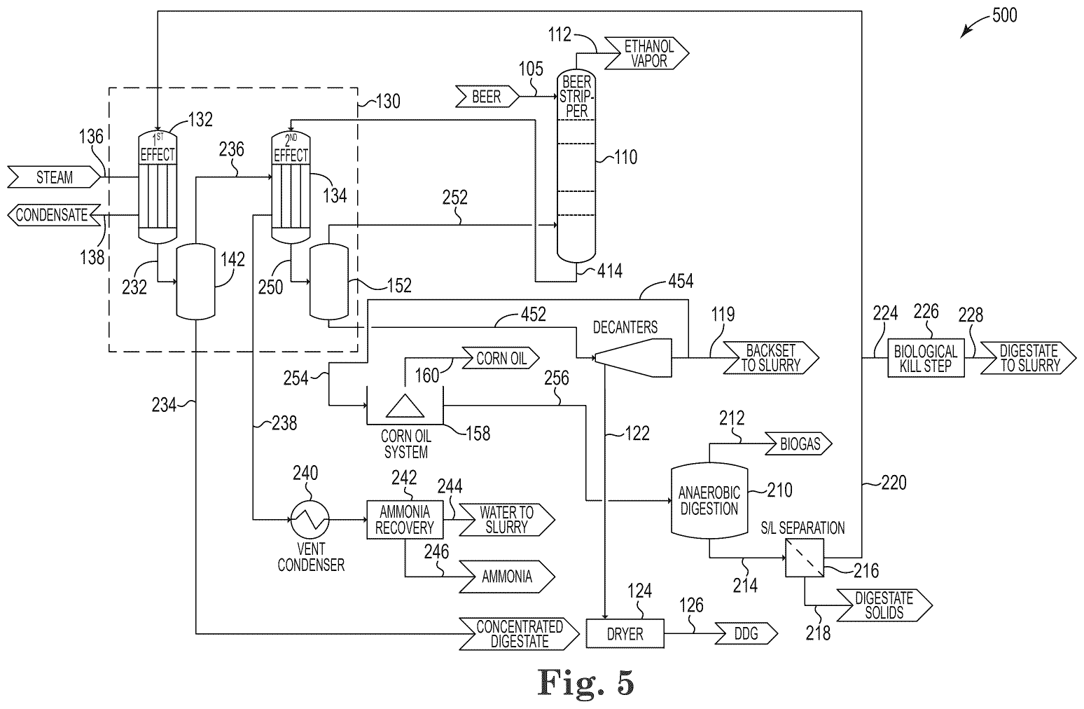

FIG. 5 shows another alternative bioprocessing facility 500 that incorporates anaerobic digestion. Bioprocessing facility 500 is similar to bioprocessing facility 200 in FIG. 2, described above, except that wet cake 122 is dried in dryer system 124 to produce dried distillers' grain (DDG) 326 instead of being sent to mix tank 205 and anaerobic digestion system 210, and whole stillage 414 is sent to final-effect evaporator 134 instead of first being separated into thin stillage 118 and wet cake 122.

What is claimed is:

1. A bioprocessing facility comprising:
a source of one or more stillage compositions;
a multi-effect evaporator system comprising at least a final-effect evaporator, wherein the final-effect evaporator is configured to receive at least a portion of at least one stillage composition as feed, wherein the final-effect evaporator is configured to produce a vapor composition and a concentrated stillage composition; and
an anaerobic digestion system in fluid communication with the final-effect evaporator, wherein the anaerobic digestion system is configured to receive and digest at least a portion of the concentrated stillage composition to produce a biogas and an anaerobic digestion digestate composition, wherein the multi-effect evaporator system comprises at least one effect evaporator prior to the final-effect evaporator that is configured to receive at least a portion of the anaerobic digestion digestate composition as feed.

2. The bioprocessing facility of claim 1, further comprising a separation system in fluid communication with the anaerobic digestion digestate composition, wherein the separation system is configured to separate the anaerobic digestion digestate composition into at least an anaerobic digestion liquid effluent and an anaerobic digestion solid effluent, and wherein the multi-effect evaporator system comprises at least one effect evaporator prior to the final-effect evaporator that is configured to receive at least a portion of the anaerobic digestion liquid effluent as feed and to form a vapor composition and a concentrated anaerobic digestion digestate composition.

3. The bioprocessing facility of claim 2, wherein the at least one effect evaporator prior to the final-effect evaporator is in fluid communication with a downstream effect evaporator, wherein the downstream effect evaporator is configured to receive at least a portion of the vapor composition as steam and form a condensate comprising ammonia.

4. The bioprocessing facility of claim 3, wherein the downstream effect evaporator comprises the final-effect evaporator.

5. The bioprocessing facility of claim 3, further comprising an ammonia separation system configured to receive at least a portion of the condensate and separate at least a portion of the ammonia from the condensate.

6. The bioprocessing facility of claim 1, wherein the at least one stillage composition comprises thin stillage from a dry-grind corn ethanol facility, and wherein the concentrated stillage composition comprises concentrated thin stillage.

7. The bioprocessing facility of claim 6, further comprising a separator system in fluid communication with the concentrated thin stillage, wherein the separation system is configured to separate corn oil form the concentrated thin stillage prior to the anaerobic digestion system.

8. The bioprocessing facility of claim 6, wherein the anaerobic digestion system is configured to also receive and digest at least a portion of wet cake from a dry-grind corn ethanol facility.

9. The bioprocessing facility of claim 8, further comprising a mix tank configured to receive and mix the wet cake and the concentrated thin stillage prior to the anaerobic digestion system.

10. The bioprocessing facility of claim 1, wherein the at least one stillage composition comprises whole stillage from a dry-grind corn ethanol facility.

11. A method of producing biogas from one or more stillage compositions, wherein the method comprises:
introducing at least a portion of at least one stillage composition as feed into a final-effect evaporator to form a vapor composition and a concentrated stillage composition;
exposing at least a portion of the concentrated stillage composition to anaerobic digestion conditions to produce a biogas and an anaerobic digestion digestate composition;
and
introducing at least a portion of the anaerobic digestion digestate composition as feed into at least one effect evaporator prior to the final-effect evaporator to form a vapor composition and a concentrated anaerobic digestion digestate composition.

12. The method of claim 11, further comprising separating at least an anaerobic digestion liquid effluent from the anaerobic digestion digestate composition, wherein the introducing at least a portion of the anaerobic digestion digestate composition comprises introducing at least a portion of the anaerobic digestion liquid effluent as feed into at least one effect evaporator prior to the final-effect evaporator to form a vapor composition and a concentrated anaerobic digestion liquid effluent.

13. The method of claim 11, wherein the at least one stillage composition is derived from a grain feedstock, and further comprising separating at least a portion of grain oil from the concentrated stillage composition prior to exposing the at least a portion of the concentrated stillage composition to anaerobic digestion conditions.

14. The method of claim 11, further comprising introducing at least a portion of the vapor composition into a distillation system to heat at least a portion of contents in the distillation system, wherein the at least one stillage composition comprises whole stillage that is discharged from the distillation system.

15. The method of claim 11, wherein the vapor composition formed from the at least one effect evaporator prior to the final-effect evaporator is introduced as steam into a downstream effect evaporator to form a condensate comprising ammonia.

16. The method of claim 15, wherein the downstream effect evaporator comprises the final-effect evaporator.

17. The method of claim 16, further comprising separating at least a portion of the ammonia from the condensate.

18. The method of claim 11, wherein the anaerobic digestion digestate composition is separated into at least the anaerobic digestion liquid effluent and an anaerobic digestion solid effluent.

19. The method of claim 11, wherein the at least one stillage composition comprises thin stillage from a dry-grind corn ethanol facility, and wherein the concentrated stillage composition comprises concentrated thin stillage.

20. The method of claim 19, further comprising separating at least a portion of corn oil from the concentrated thin stillage prior to exposing the at least a portion of the concentrated thin stillage to anaerobic digestion conditions.
